(19) United States
(12) Patent Application Publication
(10) Pub. No.: US 2022/0213478 A1
(43) Pub. Date: Jul. 7, 2022

(54) ANTISENSE OLIGONUCLEOTIDES FOR THE TREATMENT OF USHER SYNDROME

(71) Applicant: ProQR Therapeutics II B.V., Leiden (NL)

(72) Inventors: Jim Swildens, Leiden (NL); Maarten Holkers, Leiden (NL); Sunseeahray Eugenie Elizabeth Naomi Mahakena, Leiden (NL); Kalyana Chakravarthi Dulla, Leiden (NL); Hee Lam Chan, Leiden (NL)

(21) Appl. No.: 17/604,335

(22) PCT Filed: Apr. 17, 2020
(86) PCT No.: PCT/EP2020/060854

§ 371 Date: Oct. 15, 2021

(30) Foreign Application Priority Data
Apr. 18, 2019 (EP) 19 17 0001.2
Oct. 16, 2019 (EP) 19 20 3681.2

(51) Int. Cl. C12N 15/113; C12N 15/11

(52) U.S. Cl. CPC C12N 15/113; C12N 15/111; C12N 2310/11; C12N 2320/33; C12N 2310/322; C12N 2310/3525; C12N 2310/315

(57) ABSTRACT

The invention relates to the fields of medicine. In particular, it relates to novel antisense oligonucleotides (AONs) that are capable of skipping exon 62 from human USH2A premRNA and that may be used in the treatment, prevention and/or delay of Usher syndrome type II and/or USH2A-associated non syndromic retina degeneration.

### FIELD OF THE INVENTION

The invention relates to the field of medicine. In particular, it relates to single-stranded antisense oligonucleotides (AONs) for use in the treatment, prevention and/or delay of eye diseases, preferably Usher syndrome, and/or USH2A-associated retinal degeneration.

### BACKGROUND OF THE INVENTION

Usher syndrome (USH, or just ‘Usher’) and non-syndromic retinitis pigmentosa (NSRP) are degenerative diseases of the retina. Usher is clinically and genetically heterogeneous and by far the most common type of inherited deaf-blindness in man (1 in 6,000 individuals; Kimberling et al. 2010. Genet Med 12:512-516). The hearing impairment in Usher patients is mostly stable and congenital and can be partly compensated by hearing aids or cochlear implants. The degeneration of photoreceptor cells in Usher and NSRP is progressive and often leads to complete blindness between the third and fourth decade of life, thereby leaving time for therapeutic intervention. Mutations in the USH2A gene are the most frequent cause of Usher syndrome type IIa explaining up to 50% of all Usher patients worldwide (±1300 patients in the Netherlands) and, as indicated by McGee et al. (2010. J Med Genet 47(7):499-506), also the most prevalent cause of NSRP in the USA, likely accounting for 12-25% of all cases of retinitis pigmentosa (RP). The mutations are spread throughout the seventy-two USH2A exons and their flanking intron sequences, and consist of nonsense and missense mutations, deletions, duplications, large rearrangements, and splicing variants. Exon 13 is by far the most frequently mutated exon with two founder mutations (c.2299deIG (p.E767SfsX21) in USH2 patients and c.2276G>T (p.C759F) in NSRP patients). For exon 50, fifteen pathogenic mutations have been reported, of which at least eight are clearly protein-truncating. Also, a deep-intronic mutation in intron 40 of USH2A (c.7595-2144A>G) was reported (Vache et al. 2012. Human Mutation 33(1):104-108), which creates a cryptic high-quality splice donor site in intron 40 resulting in the inclusion of an aberrant exon of 152 bp (Pseudo Exon 40, or PE40) in the mutant USH2A mRNA, that causes premature termination of translation.

Usher and other retinal dystrophies have for long been considered as incurable disorders. Several phase I/II clinical trials using gene augmentation therapy have led to promising results in selected groups of LCA/RP/USH patients with mutations in the RPE65 gene (Bainbridge et al. 2008. N Engl J Med 358, 2231-2239) and MYO7A gene (Hashimoto et al. 2007. Gene Ther 14(7):584-594). The size of the coding sequence (15,606 bp) and alternative splicing of the USH2A gene and mRNA hamper gene augmentation therapy due to the currently limiting cargo size of many available vectors (such as adeno-associated virus (AAV) and lentiviral vectors).

Over the last decade several antisense oligonucleotide (AON)-based therapies for the eye have been developed (WO2012/168435; WO2013/036105; WO2015/004133; WO2016/005514; WO2016/034680; WO2016/135334; WO2017/060317; WO2017/186739; WO2018/055134; WO2018/189376), with a mutated CEP290-targeting product (sepofarsen, for Leber's Congenital Amaurosis type 10, or LCA10) and a mutated USH2A exon 13 targeting AON (QR-421a for Usher syndrome and NSRP) proceeding into clinical trials showing very promising effects. AONs are generally small polynucleotide molecules (16- to 25-mers) that are able to interfere with splicing as their sequence is complementary to that of target pre-mRNA molecules. The envisioned mechanism is such that upon binding of an AON to a target sequence, with which it is complementary, the targeted region within the pre-mRNA is no longer available for splicing factors which in turn results in skipping of the targeted exon. Therapeutically, this methodology can be used in two ways: a) to redirect normal splicing of genes in which mutations activate cryptic splice sites and b) to skip exons that carry mutations such that the reading frame of the mRNA remains intact and a (partially or fully) functional protein is made. For the USH2A gene, 28 out of the 72 described exons can potentially be skipped without disturbing the overall reading frame of the transcript. These in-frame exons include exon 13 and 50. WO2016/005514 discloses exon skipping AONs for the USH2A pre-mRNA, directed at skipping of exon 13, exon 50 and PE40. WO2017/186739 discloses PE40 skipping AONs and WO2018/055134 discloses exon 13 skipping AONs.

Clearly, there is a need for additional and alternative AONs that would affect splicing events elsewhere in the USH2A pre-mRNA and cause the skip of other in-frame exons, while then restoring (at least partially) the usherin function, which is the protein encoded by the USH2A gene. One other exon in the human USH2A gene that was found to be often mutated is exon 62, with reports disclosing the pathogenic mutations c.12093del, c.12234_12235del, c.12172_12174delinsTAAA, c.12175dup and c.12274del (Bonnet et al. 2016. Eur J Hum Genet 24:1730-178; Aparisi et al. 2014. Orph J Rare Dis 9:168; Baux et al. 2007. Hum Mut 28(8):781-789). Based on these reports it is estimated that there are +/−650 patients with pathogenic exon 62 mutations in the western world. It is an objective of the present invention to provide AONs that can be used in a convenient therapeutic strategy for the prevention, treatment or delay of Usher and/or NSRP caused by mutations in exon 62 of the human USH2A gene.

### SUMMARY OF THE INVENTION

The present invention relates to an antisense oligonucleotide (AON) capable of skipping exon 62 from human USH2A pre-mRNA, wherein the AON under physiological conditions binds to and/or is complementary to a sequence of SEQ ID NO: 25 or 26, or a part thereof. In a preferred embodiment, the AON of the present invention, under physiological conditions binds to and/or is complementary to a sequence of SEQ ID NO: 25 that includes the 5′ intron/exon boundary of exon 62. In another preferred embodiment, the AON of the present invention, under physiological conditions binds to and/or is complementary to a sequence of SEQ ID NO: 25 wherein the complementary sequence is completely within exon 62. In another preferred embodiment, the AON of the present invention, under physiological conditions binds to and/or is complementary to a sequence of SEQ ID NO: 25 that includes the 3′ exon/intron boundary of exon 62. Preferably, the AON of the present invention is an oligoribonucleotide. In one particularly preferred aspect, the AON according to the invention comprises at least one 2′-O-methoxyethyl (2′-MOE) modification. More preferably, all nucleotides of the AON are 2′-MOE modified. In yet another preferred embodiment, the AON according to the invention comprises at least one non-naturally occurring internucleoside linkage, such as a phosphorothioate (PS) linkage, more preferably, wherein all sequential nucleosides are interconnected by PS linkages.

In another embodiment, the invention relates to a viral vector expressing an AON according to the invention. In another embodiment, the invention relates to a pharmaceutical composition comprising an AON according to the invention, or a viral vector according to the invention, and a pharmaceutically acceptable carrier.

In another embodiment, the invention relates to an AON according to the invention, a viral vector according to the invention, or a pharmaceutical composition according to the invention for use in the treatment, prevention or delay of an USH2A-related disease or a condition requiring modulating splicing of USH2A pre-mRNA, such as Usher syndrome type II.

The invention also relates to a use of an AON according to the invention, a viral vector according to the invention, or a pharmaceutical composition according to the invention for the preparation of a medicament for the treatment, prevention or delay of an USH2A-related disease or a condition requiring modulating splicing of USH2A pre-mRNA, such as Usher syndrome type II.

The invention furthermore relates to a method for the treatment of a USH2A-related disease or condition requiring modulating splicing of USH2A pre-mRNA of an individual in need thereof, said method comprising contacting a cell of said individual with an AON according to the invention, a viral vector according to the invention, or a pharmaceutical composition according to the invention.

### BRIEF DESCRIPTION OF THE DRAWINGS

FIG. 1 shows the 5′ to 3′ DNA sequence of exon 62 (in bold, upper case) plus its flanking intron sequences (lower case). (A) shows the first part, (B) shows the second part and (C) shows the third part of this continuous sequence. The sequence of exon 62 with its flanking sequences as shown here is provided as SEQ ID NO: 59, which represents the DNA sequence as present in the gene, but that represents the RNA sequence when transcribed into pre-mRNA. A longer sequence (with 20 additional nucleotides of intron 61, upstream of exon 62) is provided as SEQ ID NO: 23. The corresponding RNA sequence of SEQ ID NO: 23 is provided herein as SEQ ID NO: 25. The coding DNA sequence of exon 62 without flanking sequences is provided as SEQ ID NO: 24, whereas the corresponding RNA sequence is provided as SEQ ID NO: 26. Shown here are also the sequences of the forty-eight AONs described herein (3′ to 5′; AON Ex62.1 to AON Ex62.48; provided in that order in SEQ ID NO: 1 to 22 and SEQ ID NO: 33 to 58) and their position in relation to the target sequence. AON Ex62.49, -50, -51, and -52 are SEQ ID NO: 60, 61, 62, and 63 respectively.

FIG. 2 shows the percentage of exon 62 skip as determined by digital droplet PCR (ddPCR) after a transfection of twenty-two AONs (given by their abbreviated names below the graph) in human retinoblastoma cells. No transfection (NT) and mock transfections served as negative controls. The order of the AONs from left to right represents their position towards their complementary target sequence from 5′ to 3′ in SEQ ID NO: 59.

FIG. 3 shows the percentage of exon 62 skip as determined by ddPCR after transfection of a next set of AONs. The order of the AONs from left to right represent their position towards their complementary target sequence (see FIG. 1). Black bars represent AONs that were tested in the experiment of FIG. 2. Open bars represent newly tested AONs.

FIG. 4 shows the percentage of exon 62 skip as determined by ddPCR after transfection (A) and gymnotic uptake (B) of a new set of AONs comprising nineteen AONs covering two hot spot areas as detailed in FIG. 1 and the examples. The order of the AONs from left to right represent their position towards their complementary target sequence (see FIG. 1). Black bars represent AONs that were tested in the experiment of FIG. 2 and/or 3. Open bars represent newly tested AONs.

FIG. 5 shows the percentage of exon 62 skip as determined by ddPCR after gymnotic uptake of the AONs mentioned below the graph. All AONs were fully 2′-MOE modified and AONs Ex62.44, -45, -46, -47, and -48 were tested for the first time in this experiment. The length of the oligonucleotides is given above the bars. The order of the AONs from left to right represent their position towards their complementary target sequence (see FIG. 1).

FIG. 6 shows the percentage of exon 62 skip as determined by ddPCR after gymnotic uptake of the AONs mentioned below the graph. On the left the results with the AONs modified with 2′-MOE are shown, while on the right the results of some (but not all) of the corresponding AONs modified with 2′-OMe are shown. AON Ex62.49 (SEQ ID NO: 60) was newly tested.

FIG. 7 shows the percentage of exon 62 skip as determined by ddPCR after gymnotic uptake of four oligonucleotides, all fully modified with 2′-MOE: AON Ex62.34 (A), AON Ex62.46 (B), AON Ex62.48 (C) and AON Ex62.49 (D). Four different concentrations were used, as depicted.

FIG. 8 shows the percentage of exon 62 skip as determined by ddPCR after administering four different AONs to eyecups (organoids) cultured from human cells, as outlined in the examples. All four tested AONs were fully 2′-MOE modified and used in two different concentrations, as shown.

### DETAILED DESCRIPTION

The present invention relates to specific antisense oligonucleotides (AONs) that can block the inclusion of exon 62 in human USH2A mRNA. More specifically, the present invention relates to an AON for skipping exon 62 in human USH2A pre-mRNA, wherein the AON under physiological conditions binds to and/or is complementary to the sequence of SEQ ID NO: 25, or a part thereof. In a preferred embodiment, the invention relates to an AON capable of skipping exon 62 from human USH2A pre-mRNA, wherein the AON under physiological conditions binds to and/or is complementary to a sequence that includes the intron/exon boundary at the 5′ end of exon 62 of the human USH2A gene. In another preferred embodiment, the present invention relates to an AON capable of skipping exon 62 from human USH2A pre-mRNA, wherein the AON comprises or consists of the sequence of SEQ ID NO: 1, 2, 3, 4, 5, 6, 7, 8, 9, 10, 11, 12, 13, 14, 15, 16, 17, 18, 19, 20, 21, 22, 33, 34, 35, 36, 37, 38, 39, 40, 41, 42, 43, 44, 45, 46, 47, 48, 49, 50, 51, 52, 53, 54, 55, 56, 57, 58, 60, 61, 62 or 63.

In a preferred aspect said AON is an oligoribonucleotide. In a further preferred aspect, the AON according to the invention comprises a 2′-O alkyl modification, such as a 2′-O-methyl (2′-OMe) modified sugar. In a more preferred embodiment, all nucleotides in the AON are 2′-OMe modified. In another preferred aspect, the invention relates to an AON comprising a 2′-O-methoxyethyl (2′-methoxyethoxy, or 2′-MOE) modification. In a more preferred embodiment, all nucleotides of said AON carry a 2′-MOE modification. In yet another aspect the invention relates to an AON comprising at least one 2′-OMe and at least one 2′-MOE modification. In another preferred embodiment, the AON according to the present invention comprises at least one phosphorothioate (PS) modified linkage. In another preferred aspect, all sequential nucleotides are interconnected by PS linkages.

In yet another aspect, the invention relates to a viral vector expressing an AON according to the invention. The invention also relates to a pharmaceutical composition comprising an AON according to the invention or a viral vector according to the invention, and a pharmaceutically acceptable carrier.

In another embodiment, the invention relates to an AON according to the invention, a viral vector according to the invention, or a pharmaceutical composition according to the invention for use in the treatment, prevention or delay of an USH2A-related disease or a condition requiring modulating splicing of USH2A pre-mRNA, such as Usher syndrome type II. A preferred USH2A-related disease or condition is one that is caused by a mutation in exon 62 of the human USH2A gene. In one aspect, the invention relates to an AON for use according to the invention, wherein the AON is for intravitreal administration and is dosed in an amount ranging from 5 μg to 500 μg of total AON per eye, preferably from 10 μg to 100 μg, more preferably from 25 μg to 100 μg. Preferably, the AON is administered in a naked form (as is, without being carried by a particle such as a nanoparticle or liposome), and preferably the administration to the vitreous is by direct injection. Preferably, the AON for use according to the invention is administered to the eye, wherein the AON is dosed in an amount ranging from 5 μg to 500 μg of total AON per eye, such as about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, or 320 μg total AON per eye.

In another embodiment the invention relates to a use of an AON according to the invention, a viral vector according to the invention, or a pharmaceutical composition according to the invention for the preparation of a medicament for the treatment, prevention or delay of an USH2A-related disease or a condition requiring modulating splicing of USH2A pre-mRNA, such as Usher syndrome type II.

In another embodiment, the invention relates to an in vitro, ex vivo or in vivo method for modulating splicing of USH2A pre-mRNA in a cell, comprising the steps of: administering to the cell an AON according to the invention, a viral vector according to the invention, or a pharmaceutical composition according to the invention; allowing the hybridization of the AON to its complementary sequence in USH2A target RNA molecule in the cell; and allowing the skip of exon 62 from the target RNA molecule. Optionally, the method further comprises the step of analyzing whether the skip of exon 62 from the USH2A target RNA molecule has occurred, which can be performed using methods as disclosed herein and/or by other methods generally known to the person skilled in the art. The invention also relates to a method for the treatment of a USH2A-related disease or condition requiring modulating splicing of USH2A pre-mRNA of an individual in need thereof, said method comprising contacting a cell of said individual with an AON according to the invention, a viral vector according to the invention, or a pharmaceutical composition according to the invention. Contacting the cell of the individual may be in vivo, by direct intravitreal administration of the AON to the patient in need thereof, or through ex vivo procedures, wherein treated cells, that have received the AON, viral vector or pharmaceutical composition, are transplanted back to the patient, thereby to treat the disease.

In all embodiments of the invention, the terms ‘modulating splicing’ and ‘exon skipping’ are synonymous. In respect of USH2A, ‘splice switching’, ‘modulating splicing’ or ‘exon skipping’ are to be construed as the exclusion of exon 62 from the resulting USH2A mRNA. In a preferred setting the exon 62 that needs to be skipped harbors unwanted mutations, leading to Usher syndrome. For the purpose of the invention the terms ‘aberrant exon 62’ or ‘aberrant USH2A exon 62’ are synonymous and considered to mean the presence of a mutation in exon 62 of the human USH2A gene.

The term ‘exon skipping’ is herein defined as inducing, producing or increasing production within a cell of a mature mRNA that does not contain a particular exon (in the current case exon 62 of the human USH2A gene) that would be present in the mature mRNA without exon skipping. Exon skipping is achieved by providing a cell expressing the pre-mRNA of said mature mRNA with a molecule capable of interfering with sequences such as, for example, the (cryptic) splice donor or (cryptic) splice acceptor sequence required for allowing the enzymatic process of splicing, or with a molecule that is capable of interfering with an exon inclusion signal required for recognition of a stretch of nucleotides as an exon to be included in the mature mRNA; such molecules are herein referred to as ‘exon skipping molecules’, as ‘exon 62 skipping molecules’, as ‘AONs capable of skipping exon 62 from human USH2A pre-mRNA’, or as ‘exon skipping AONs’, and varieties thereof. The term ‘pre-mRNA’ refers to a non-processed or partly processed precursor mRNA that is synthesized from a DNA template of a cell by transcription, such as in the nucleus.

The terms ‘antisense oligonucleotide’, ‘oligonucleotide’, single-stranded antisense oligonucleotide’, ‘AON’, and varieties thereof are understood to refer to a molecule with a nucleotide sequence that is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. The terms ‘antisense oligonucleotide’, ‘oligonucleotide’, ‘AON’ and ‘oligo’ are used interchangeably herein and are understood to refer to an oligonucleotide comprising an antisense sequence in respect of the target RNA (or DNA) sequence.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 0.1% of the value.

In one embodiment, an exon 62 skipping molecule as defined herein is an AON that binds and/or is complementary to a specified target RNA sequence within a target RNA molecule, preferably a target pre-mRNA molecule. Binding to one of the specified target sequences, preferably in the context of a mutated USH2A exon 62 may be assessed via techniques known to the skilled person. A preferred technique is gel mobility shift assay as described in EP1619249. In a preferred embodiment, an exon 62 skipping AON is said to bind to one of the specified sequences as soon as a binding of said molecule to a labeled target sequence is detectable in a gel mobility shift assay.

In all embodiments of the invention, an exon 62 skipping molecule is preferably an AON. Preferably, an exon 62 skipping AON according to the invention is an AON, which is complementary or substantially complementary to a nucleotide sequence of SEQ ID NO: 25, or a part thereof.

The term ‘substantially complementary’ used in the context of the invention indicates that some mismatches in the antisense sequence are allowed as long as the functionality, i.e. inducing skipping of the mutated USH2A exon 62 is still acceptable. Preferably, the complementarity is from 90% to 100%. In general, this allows for 1 or 2 mismatches in an AON of 20 nucleotides or 1, 2, 3 or 4 mismatches in an AON of 40 nucleotides, or 1, 2, 3, 4, 5, or 6 mismatches in an AON of 60 nucleotides, etc. The skilled person understands that an AON may be 100% complementary to a sequence harboring a mutation, which means that it is not 100% complementary to the corresponding wild type sequence, while it is still active in causing exon 62 skipping in both wild type and mutant settings. This means that the AONs as disclosed herein, and which are 100% complementary to the wild type USH2A sequence, may be used in a slightly modified form to become 100% complementary to the mutant sequence, when the mutation is in the complementary stretch of the AON. The invention therefore also relates to the AONs that are modified to become 100% complementary to the mutant sequence, although a complementarity that is not 100% (to the wild type or the mutant sequence) is not explicitly excluded, when such AON may have additional beneficial properties (higher stability, better efficiency, etc., based on what has been disclosed by the present invention.

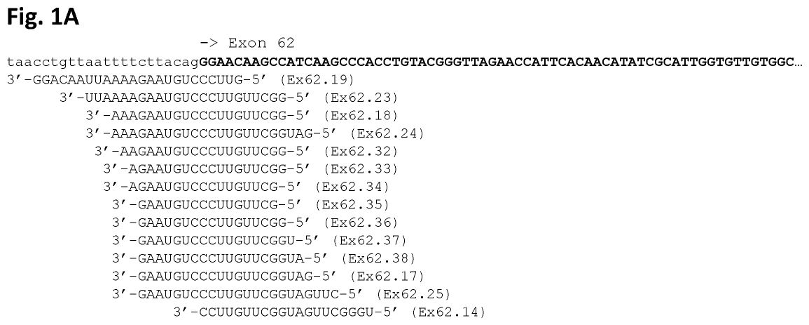

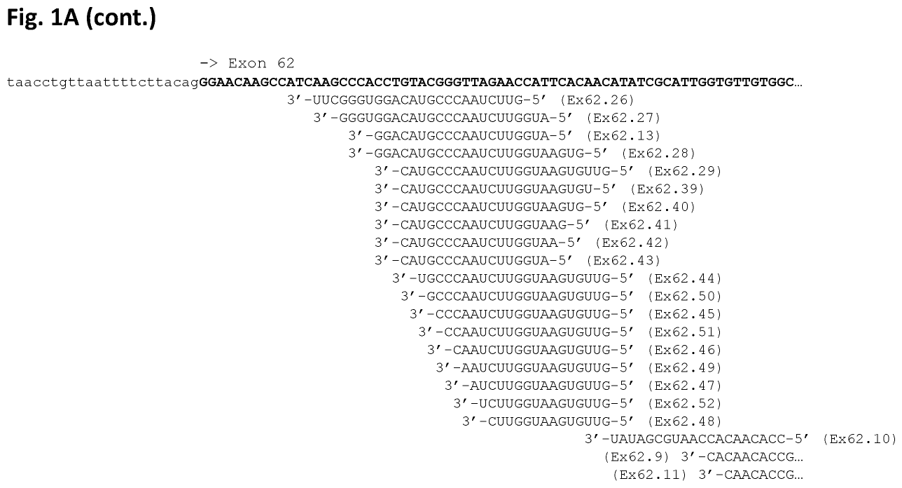

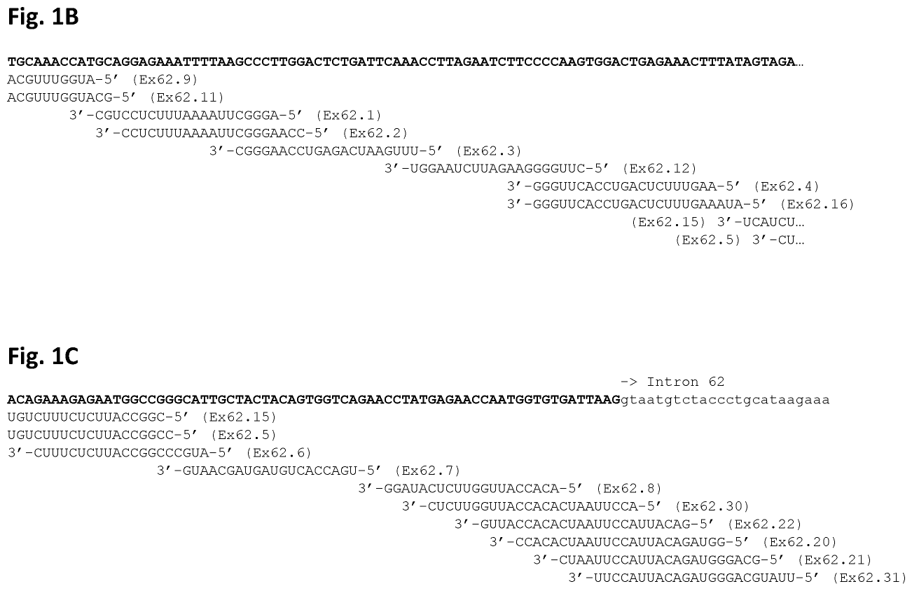

The invention provides a method for designing an exon 62 skipping AON able to induce skipping of the mutated USH2A exon 62. First, the AON is selected to bind to and/or to be complementary to exon 62, possibly with stretches of the flanking intron sequences as shown in SEQ ID NO: 25 or 59 (see FIG. 1). Subsequently, in a preferred method at least one of the following aspects has to be taken into account for designing, improving said exon skipping AON further: the exon skipping AON preferably does not contain a CpG or a stretch of CpG; and the exon skipping AON has acceptable RNA binding kinetics and/or thermodynamic properties. The presence of a CpG or a stretch of CpG in an AON is usually associated with an increased immunogenicity of said AON (Dorn and Kippenberger. 2008. Curr Opin Mol Ther 10(1):10-20). This increased immunogenicity is undesired since it may induce damage of the tissue to be treated, i.e. the eye. Immunogenicity may be assessed in an animal model by assessing the presence of CD4+and/or CD8+cells and/or inflammatory mononucleocyte infiltration. Immunogenicity may also be assessed in blood of an animal or of a human being treated with an AON of the invention by detecting the presence of a neutralizing antibody and/or an antibody recognizing said AON using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said AON using a standard immunoassay.

The invention allows designing an AON with acceptable RNA binding kinetics and/or thermodynamic properties. The RNA binding kinetics and/or thermodynamic properties are at least in part determined by the melting temperature of an AON (Tm), and/or the free energy of the AON-target exon complex, applying methods known to the person skilled in the art. If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol.

An AON of the invention is preferably one that can exhibit an acceptable level of functional activity. A functional activity of said AON is preferably to induce the skipping of the mutant USH2A exon 62 to a certain acceptable level, to provide an individual with a functional usherin protein and/or USH2A mRNA and/or at least in part decreasing the production of an aberrant usherin protein and/or mRNA. In a preferred embodiment, an AON is said to induce skipping of the mutated USH2A exon 62, when the mutated USH2A exon 62 skipping percentage as measured by digital-droplet PCR (ddPCR) is at least 5%, or at least 10%, or at least 15%, or at least 20%, or at least 50%, or at least 55%, or at least 60%, or at least 65%, or at least 70%, or at least 75%, or at least 80%, or at least 85%, or at least 90%, or at least 95%, or 100% as compared to a control RNA product not treated with an AON or a negative control AON. Assays to determine exon skipping and/or exon retention are described in the examples herein and may be supplemented with techniques known to the person skilled in the art.

Preferably, an AON, which comprises a sequence that is complementary or substantially complementary to a nucleotide sequence of SEQ ID NO: 25 of USH2A is such that the (substantially) complementary part is at least 50% of the length of the AON according to the invention, more preferably at least 60%, even more preferably at least 70%, even more preferably at least 80%, even more preferably at least 90% or even more preferably at least 95%, or even more preferably 98% or even more preferably at least 99%, or even more preferably 100%. Preferably, an AON according to the invention comprises or consists of a sequence that is complementary to SEQ ID NO: 25, or a part thereof.

In another preferred embodiment, the length of said complementary part of said AON is at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 115, 120, 125, 130, 135, 140, 141, 142 or 143 nucleotides. Additional flanking sequences may be used to modify the binding of a protein to the AON, or to modify a thermodynamic property of the AON, more preferably to modify target RNA binding affinity.

As stated, it is thus not absolutely required that all the bases in the region of complementarity are capable of pairing with bases in the opposing strand. For instance, when designing the AON, one may want to incorporate for instance a residue that does not base pair with the base on the complementary strand. Mismatches may, to some extent, be allowed, if under the circumstances in the cell, the stretch of nucleotides is sufficiently capable of hybridizing to the complementary part. In this context, ‘sufficiently’ preferably means that using a gel mobility shift assay as described in example 1 of EP1619249, binding of an AON is detectable.

Optionally, said AON may further be tested by transfection into retina cells of patients, by delivering the AONs directly to so-called eye-cups, which are ex vivo generated eye models (generally generated from patient's cells), directly to organoids, or by direct intravitreal injection in an animal model, or by direct intravitreal administration in human patients in the course of performing clinical trials. Testing of AONs in eyecups is exemplified in the accompanying examples. Skipping of targeted exon 62 may be assessed by RT-PCR or by ddPCR. The complementary regions are preferably designed such that, when combined, they are specific for the exon in the pre-mRNA. Such specificity may be created with various lengths of complementary regions as this depends on the actual sequences in other (pre-) mRNA molecules in the system. The risk that the AON also will be able to hybridize to one or more other pre-mRNA molecules decreases with increasing size of the AON. It is clear that AONs comprising mismatches in the region of complementarity but that retain the capacity to hybridize and/or bind to the targeted region(s) in the pre-mRNA, can be used in the invention. However, preferably at least the complementary parts do not comprise such mismatches as AONs lacking mismatches in the complementary part typically have a higher efficiency and a higher specificity, than AONs having such mismatches in one or more complementary regions. It is thought that higher hybridization strengths (i.e. increasing number of interactions with the opposing strand) are favorable in increasing the efficiency of the process of interfering with the splicing machinery of the system. Preferably, the complementarity is from 90% to 100%.

An exon skipping AON of the invention is preferably an isolated single stranded molecule in the absence of its (target) counterpart sequence. An exon skipping AON of the invention is preferably complementary to, or under physiological conditions binds to a sequence present within SEQ ID NO: 25, more preferably where it is complementary to a region that overlaps with the 5′ intron/exon boundary of exon 62 (such as AON Ex62.34), where it is complementary to a stretch of oligonucleotides surrounding the area that is targeted by AON Ex62.44, such as seen in the accompanying examples with AON Ex62.45 to -49 and anticipated to be seen with AON Ex62.50 to -52, or with the 3′ exon/intron boundary of exon 62. If an AON is complementary to a sequence that includes either one of these boundaries, this means that at least the last nucleotide of the upstream (5′) located intron and the first nucleotide of the exon are included in the complementary region, and on the other side of the exon, it means that at least the last nucleotide of the exon and the first nucleotide of the downstream (3′) intron are included in the complementary region. It will be understood that an exon 62 skipping AON does not have to be complementary to the sequence in exon 62 that is mutated. It may be that the AON is complementary to the wild type exon 62 sequence and/or its surrounding intron sequences, while still being able to give exon 62 skipping. The aim is to skip a mutated exon 62 from USH2A pre-mRNA, not to have an AON that specifically targets a region containing the mutation in exon 62, although such is not explicitly excluded. Any mutation in USH2A exon 62 that causes disease (such as Usher syndrome) is preferably removed from the final mRNA (and the resulting protein) by using an AON of the present invention, wherein the sequence of the AON may be complementary to a non-mutated region. The invention also relates to AONs that may be fully complementary to the wild type target sequence but may also be adjusted in sequence to become 100% complementary to a mutant sequence, if the mutation is in the region of AON complementarity, as outlined above. In that case the AON is substantially complementary to the mutant sequence and may then differ from the wild type sequences of the AONs that are generally referred to herein. The invention is generally explained for any mutation that may be present in the USH2A exon 62 sequence, but specific mutations may be targeted by AONs that are (preferably 100%) complementary to that specific mutation and its surrounding sequences, 5′ and/or 3′ from the mutation.

A preferred exon 62 skipping AON of the invention comprises or consists of from 8 to 143 nucleotides, more preferably from 10 to 40 nucleotides, more preferably from 12 to 30 nucleotides, more preferably from 14 to 30 nucleotides, more preferably 17 to 21 nucleotides. An AON according to the present invention preferably consists of 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 115, 120, 125, 130, 135, 140, 141, 142 or 143 nucleotides. Most preferably, the exon 62 skipping AON of the invention consists of 14, 15, 16, 17, 18, 19, 20, 21, 22, 23, or 24 nucleotides, and more preferably consists of 14, 15, 16, 17, 18, 19, or 20 nucleotides.

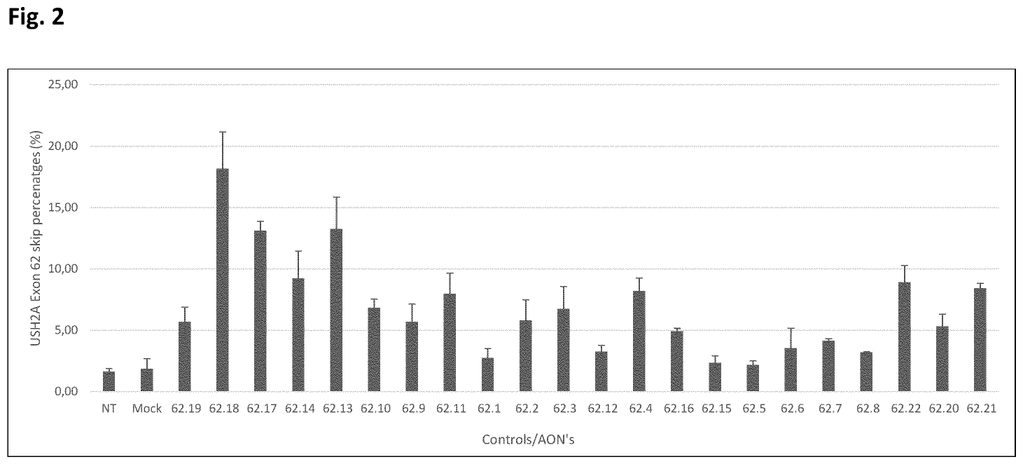

In certain embodiments, the invention provides an exon 62 skipping AON selected from the group consisting of SEQ ID NO: 1, 2, 3, 4, 5, 6, 7, 8, 9, 10, 11, 12, 13, 14, 15, 16, 17, 18, 19, 20, 21, 22, 33, 34, 35, 36, 37, 38, 39, 40, 41, 42, 43, 44, 45, 46, 47, 48, 49, 50, 51, 52, 53, 54, 55, 56, 57, 58, 60, 61, 62, or 63. In a preferred embodiment, the invention provides an exon 62 skipping AON comprising or preferably consisting of the sequence as provided in SEQ ID NO: 13, 14, 17, 18, 20, 21, 22, 33, 34, 38, 39, 40, 41, 42, 43, 44, 45, or 46. Especially preferred are AONs that are 14, 15, 16, 17, and 18 nucleotides in length, exemplified by AON Ex62.34, -35, -36, 47, and -48 (SEQ ID NO: 44, 45, 46, 57, and 58 respectively) and AON Ex62.49 (SEQ ID NO: 60) that consists of 17 nucleotides. It was found that these molecules are very efficient in modulating splicing of the mutated USH2A exon 62 (see FIGS. 2-8), especially when they were addressed in a gymnotic uptake assessment and in the administration to eyecups, which represents naked delivery in vivo by direct intravitreal administration in the eye, without the use of delivering (or transfection) agents.

An exon 62 skipping AON according to the invention may contain one of more RNA residues, or one or more DNA residues, and/or one or more nucleotide analogues or equivalents, as will be further detailed herein below. It is preferred that an exon 62 skipping AON of the invention comprises one or more residues that are modified by non-naturally occurring modifications to increase nuclease resistance, and/or to increase the affinity of the AON for the target sequence. Therefore, in a preferred embodiment, the AON sequence comprises at least one nucleotide analogue or equivalent, wherein a nucleotide analogue or equivalent is defined as a residue having a modified base, and/or a modified backbone, and/or a non-natural internucleoside linkage, or a combination of these modifications.

### Modifications

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one aspect, the nucleobase in an AON of the present invention is adenine, cytosine, guanine, thymine, or uracil. In another aspect, the nucleobase is a modified form of adenine, cytosine, guanine, or uracil. In another aspect, the modified nucleobase is hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like. The sugar moiety can be a pyranose or derivative thereof, or a deoxypyranose or derivative thereof, preferably ribose or derivative thereof, or deoxyribose or derivative thereof. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O, 4′-C-ethylene-bridged nucleic acid (Morita et al. 2001. Nucleic Acid Res Supplement No. 1:241-242).

Sometimes the terms adenosine and adenine, guanosine and guanine, cytosine and cytidine, uracil and uridine, thymine and thymidine, inosine and hypoxanthine, are used interchangeably to refer to the corresponding nucleobase, nucleoside or nucleotide. Sometimes the terms nucleobase, nucleoside and nucleotide are used interchangeably, unless the context clearly requires differently. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

In one aspect, an AON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. An AON of the present invention comprises at least one 2′-substituted RNA monomer connected through or linked by a phosphorothioate or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. In a preferred embodiment of this aspect is provided an AON according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, any other 2′-substituted monomer within the AON is a 2′-substituted RNA monomer, such as a 2′-OMe RNA monomer or a 2′-MOE RNA monomer, which may also appear within the AON in combination.

Throughout the application, a 2′-OMe monomer within an AON of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e. the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are phosphorothioate (PS), chirally pure phosphorothioate, Rp phosphorothioate, Sp phosphorothioate, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

The present invention also relates to a chirally enriched population of modified AONs according to the invention, wherein the population is enriched for modified AONs comprising at least one particular phosphorothioate internucleoside linkage having a particular stereochemical configuration, preferably wherein the population is enriched for modified AONs comprising at least one particular phosphorothioate internucleoside linkage having the Sp configuration, or wherein the population is enriched for modified AONs comprising at least one particular phosphorothioate internucleoside linkage having the Rp configuration.

In a preferred embodiment, the nucleotide analogue or equivalent comprises a modified backbone, exemplified by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. 1991. Science 254:1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively (Egholm et al. 1993. Nature 365:566-568).

In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report demonstrated triplex formation by a morpholino oligonucleotide, and, because of the non-ionic backbone, these studies showed that the morpholino oligonucleotide was capable of triplex formation in the absence of magnesium.

In yet a further embodiment, a nucleotide analogue or equivalent of the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate (PS), chiral phosphorothioate, phosphorodithioate, phosphotriester, phosphonoacetate, aminoalkylphosphotriester, H-phosphonate, methyl and other alkyl phosphonate including methylphosphonate, 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

In another embodiment, a nucleotide analogue or equivalent of the invention comprises one or more sugar moieties that are mono- or di-substituted at the 2′, 3′ and/or 5′ position with modifications such as:
—OH;
—F;
substituted or unsubstituted, linear or branched lower (C1-010) alkyl, alkenyl, alkynyl, alkaryl, allyl, or aralkyl, that may be interrupted by one or more heteroatoms;
—O—, S—, or N-alkyl (e.g. —O-methyl);
—O—, S—, or N-alkenyl;
—O—, S—, or N-alkynyl;
—O—, S—, or N-allyl;
—O-alkyl-O-alkyl,
-methoxy;
-aminopropoxy;
-methoxyethoxy;
-dimethylamino oxyethoxy; and
-dimethylaminoethoxyethoxy.

The sugar moiety can be a pyranose or derivative thereof, or a deoxypyranose or derivative thereof, preferably ribose or derivative thereof, or deoxyribose or derivative thereof. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O,4′-C-ethylene-bridged nucleic acid (Morita et al. 2001. Nucleic Acid Res Supplement 1:241-242). These substitutions render the nucleotide analogue or equivalent RNase H and nuclease resistant and increase the affinity for the target RNA.

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by a skilled person that it is not necessary for all positions in an AON to be modified uniformly. In addition, more than one of the aforementioned analogues or equivalents may be incorporated in a single AON or even at a single position within an AON. In certain embodiments, an AON of the invention has at least two different types of analogues or equivalents. A preferred exon skipping AON according to the invention comprises a 2′-O alkyl phosphorothioated antisense oligonucleotide, such as 2′-OMe modified ribose (RNA), 2′-O-ethyl modified ribose, 2′-O-propyl modified ribose, and/or substituted derivatives of these modifications such as halogenated derivatives. An effective AON according to the invention comprises a 2′-OMe ribose and/or a 2′-MOE ribose with a (preferably full) phosphorothioated backbone.

It will also be understood by a skilled person that different AONs can be combined for efficiently skipping of the aberrant USH2A exon 62. In a preferred embodiment, a combination of at least two AONs are used in a method of the invention, such as 2, 3, 4, or 5 different AONs. Hence, the invention also relates to a set of AONs comprising at least one AON according to the present invention, optionally further comprising AONs as disclosed herein.

An AON of the present invention can be linked to a moiety that enhances uptake of the AON in cells, preferably retina cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

An exon 62 skipping AON according to the invention may be indirectly administrated using suitable means known in the art. It may for example be provided to an individual or a cell, tissue or organ of said individual in the form of an expression vector wherein the expression vector encodes a transcript comprising said oligonucleotide. The expression vector may be introduced into a cell, tissue, organ or individual via a gene delivery vehicle. In a preferred embodiment, there is provided a viral-based expression vector comprising an expression cassette or a transcription cassette that drives expression or transcription of an AON as identified herein. Accordingly, the invention provides a viral vector expressing an exon 62 skipping AON according to the invention when placed under conditions conducive to expression of the exon 62 skipping AON. A cell can be provided with an exon skipping molecule capable of interfering with essential sequences that result in highly efficient skipping of the aberrant USH2A exon 62 by plasmid-derived AON expression or viral expression provided by adenovirus- or adeno-associated virus-based vectors. Expression may be driven by a polymerase II-promoter (Pol II) such as a U7 promoter or a polymerase III (Pol III) promoter, such as a U6 RNA promoter. A preferred delivery vehicle is a viral vector such as an adeno associated virus vector (AAV), or a retroviral vector such as a lentivirus vector and the like. Also, plasmids, artificial chromosomes, plasmids usable for targeted homologous recombination and integration in the human genome of cells may be suitably applied for delivery of an oligonucleotide as defined herein. Preferred for the current invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol III driven transcripts, preferably, in the form of a fusion transcript with an U1 or U7 transcript. Such fusions may be generated as described (Gorman et al. 1998. Proc Natl Acad Sci U S A 95(9):4929-34; Suter et al. 1999. Hum Mol Genet 8(13):2415-23).

The exon 62 skipping AON may be delivered as such, or naked. However, the exon 62 skipping AON may also be encoded by the viral vector. Typically, this is in the form of an RNA transcript that comprises the sequence of an oligonucleotide according to the invention in a part of the transcript. An AAV vector according to the invention is a recombinant AAV vector and refers to an AAV vector comprising part of an AAV genome comprising an encoded exon 62 skipping AON according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. AAV vector may have one or preferably all wild type AAV genes deleted but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector. More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector. More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector. A nucleic acid molecule encoding an exon 62 skipping AON according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference. Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art. Preferably, an AAV vector according to the invention is constructed and produced according to the methods in the Examples herein. A preferred AAV vector according to the invention is an AAV vector, preferably an AAV2/5, AAV2/8, AAV2/9 or AAV2/2 vector, expressing an USH2A exon 62 skipping AON according to the invention that comprises, or preferably consists of, a sequence that is complementary or substantially complementary to a nucleotide sequence as shown in SEQ ID NO: 25, or a part thereof. A further preferred AAV vector according to the invention is an AAV vector, preferably an AAV2/5, AAV2/8, AAV2/9 or AAV2/2 vector, expressing an exon 62 skipping AON according to the invention that comprises, or preferably consists of SEQ ID NO: 1, 2, 3, 4, 5, 6, 7, 8, 9, 10, 11, 12, 13, 14, 15, 16, 17, 18, 19, 20, 21, 22, 33, 34, 35, 36, 37, 38, 39, 40, 41, 42, 43, 44, 45, 46, 47, 48, 49, 50, 51, 52, 53, 54, 55, 56, 57, 58, 60, 61, 62, or 63.

Improvements in means for providing an individual or a cell, tissue, organ of said individual with an exon 62 skipping AON according to the invention, are anticipated considering the progress that has already thus far been achieved. Such future improvements may of course be incorporated to achieve the mentioned effect on restructuring of mRNA using a method of the invention. An exon 62 skipping AON according to the invention can be delivered as is to an individual, a cell, tissue or organ of said individual. When administering an exon 62 skipping AON according to the invention, it is preferred that the AON is dissolved in a solution that is compatible with the delivery method. Retina or inner ear cells can be provided with a plasmid for AON expression by providing the plasmid in an aqueous solution. Alternatively, a preferred delivery method for an AON or a plasmid for AON expression is a viral vector or nanoparticles. Preferably viral vectors or nanoparticles are delivered to retina or inner ear cells. Such delivery to retina or inner ear cells or other relevant cells may be in vivo, in vitro or ex vivo. Nanoparticles and micro particles that may be used for in vivo AON delivery are well known in the art. Alternatively, a plasmid can be provided by transfection using known transfection reagents. For intravenous, subcutaneous, intramuscular, intrathecal and/or intraventricular administration it is preferred that the solution is a physiological salt solution. Particularly preferred in the invention is the use of an excipient or transfection reagents that will aid in delivery of each of the constituents as defined herein to a cell and/or into a cell (preferably a retina cell). Preferred are excipients or transfection reagents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), lipofectin™, DOTAP and/or viral capsid proteins that are capable of self-assembly into particles that can deliver each constituent as defined herein to a cell, preferably a retina cell. Such excipients have been shown to efficiently deliver an AON to a wide variety of cultured cells, including retina cells. Their high transfection potential is combined with an excepted low to moderate toxicity in terms of overall cell survival. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidyl ethanolamine (DOPE). The neutral component mediates the intracellular release. Another group of delivery system are polymeric nanoparticles. Polycations such as diethylamino ethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver AONs across cell membranes into cells. In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an AON. The skilled person may select and adapt any of the above or other commercially available alternative excipients and delivery systems to package and deliver an exon skipping molecule for use in the current invention to deliver it for the prevention, treatment or delay of a USH2A related disease or condition. “Prevention, treatment or delay of a USH2A related disease or condition” is herein preferably defined as preventing, halting, ceasing the progression of, or reversing partial or complete visual impairment or blindness, as well as preventing, halting, ceasing the progression of or reversing partial or complete auditory impairment or deafness that is caused by a genetic defect in the USH2A gene.

In addition, an exon 62 skipping AON according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake in to cells and/or the intracellular release of an oligonucleotide from vesicles, e.g. endosomes or lysosomes. Therefore, in a preferred embodiment, an exon 62 skipping AON according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an exon 62 skipping AON according to the invention and a further adjunct compound as defined herein. If required, an exon 62 skipping AON according to the invention or a vector, preferably a viral vector, expressing an exon 62 skipping AON according to the invention can be incorporated into a pharmaceutically active mixture by adding a pharmaceutically acceptable carrier. Accordingly, the invention also provides a composition, preferably a pharmaceutical composition, comprising an exon 62 skipping AON according to the invention, or a viral vector according to the invention and a pharmaceutically acceptable excipient. Such composition may comprise a single exon 62 skipping AON or viral vector according to the invention, but may also comprise multiple, distinct exon 62 skipping AON or viral vectors according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington (Remington. 2000. The Science and Practice of Pharmacy, 20th Edition. Baltimore, Md.: Lippincott Williams Wilkins). Each feature of said composition has earlier been defined herein.

A preferred route of administration is through direct intravitreal injection of an aqueous solution or specially adapted formulation for intraocular administration. EP2425814 discloses an oil in water emulsion especially adapted for intraocular (intravitreal) administration of peptide or nucleic acid drugs. This emulsion is less dense than the vitreous fluid, so that the emulsion floats on top of the vitreous, avoiding that the injected drug impairs vision.

If multiple distinct exon 62 skipping AONs according to the invention are used, concentration or dose defined herein may refer to the total concentration or dose of all AONs used or the concentration or dose of each exon 62 skipping AONs used or added. Therefore, in one embodiment, there is provided a composition wherein each or the total amount of exon 62 skipping AONs according to the invention used is dosed in an amount as disclosed herein.

A preferred USH2A exon 62 skipping AON according to the invention is for the treatment of an USH2A-related disease or condition of an individual. In all embodiments of the invention, the term ‘treatment’ is understood to include also the prevention and/or delay of the USH2A-related disease or condition. An individual, which may be treated using an exon 62 skipping AON according to the invention may already have been diagnosed as having a USH2A-related disease or condition. Alternatively, an individual which may be treated using an exon 62 skipping AON according to the invention may not have yet been diagnosed as having a USH2A-related disease or condition but may be an individual having an increased risk of developing a USH2A-related disease or condition in the future given his or her genetic background. A preferred individual is a human individual. In a preferred embodiment the USH2A-related disease or condition is Usher syndrome type II.

A treatment in a use or in a method according to the invention is at least once a week, once a one month, once every several months, once every 1, 2, 3, 4, 5, 6 years or longer, such as lifelong. Each exon 62 skipping AON or equivalent thereof as defined herein for use according to the invention may be suitable for direct administration to a cell, tissue and/or an organ in vivo of individuals already affected or at risk of developing USH2A-related disease or condition, and may be administered directly in vivo, ex vivo or in vitro. The frequency of administration of an AON, composition, compound or adjunct compound of the invention may depend on several parameters such as the severity of the disease, the age of the patient, the mutation of the patient, the number of exon 62 skipping AONs (i.e. dose), the formulation of said AON(s), the route of administration and so forth. The frequency may vary between daily, weekly, at least once in two weeks, or three weeks or four weeks or five weeks or a longer time period. Dose ranges of an exon 62 skipping AON according to the invention are preferably designed based on rising dose studies in clinical trials (in vivo use) for which rigorous protocol requirements exist. In a preferred embodiment, a viral vector, preferably an AAV vector as described earlier herein, as delivery vehicle for a molecule according to the invention, is administered in a dose ranging from 1×10⁹ to 1×10¹⁷ virus particles per injection, more preferably from 1×10¹⁰ to 1×10¹² virus particles per injection. The ranges of concentration or dose of AONs as given above are preferred concentrations or doses for in vivo, in vitro or ex vivo uses. The skilled person will understand that depending on the AONs used, the target cell to be treated, the gene target and its expression levels, the medium used and the transfection and incubation conditions, the concentration or dose of AONs used may further vary and may need to be optimized any further.

An exon 62 skipping AON according to the invention, or a viral vector according to the invention, or a composition according to the invention for use according to the invention may be suitable for administration to a cell, tissue and/or an organ in vivo of individuals already affected or at risk of developing a USH2A-related disease or condition, and may be administered in vivo, ex vivo or in vitro. The exon 62 skipping AON according to the invention, or viral vector according to the invention, or composition according to the invention may be directly or indirectly administered to a cell, tissue and/or an organ in vivo of an individual already affected by or at risk of developing a USH2A-related disease or condition, and may be administered directly or indirectly in vivo, ex vivo or in vitro. As Usher syndrome type II has a pronounced phenotype in retina and inner ear cells, it is preferred that said cells are retina or inner ear cells, it is further preferred that said tissue is the retina or the inner ear and/or it is further preferred that said organ is the eye or the ear. Contacting the eye or ear cell with an exon 62 skipping AON according to the invention, or a viral vector according to the invention, or a composition according to the invention may be performed by any method known by the person skilled in the art. Use of the methods for delivery of exon 62 skipping AONs, viral vectors and compositions described herein is included. Contacting may be directly or indirectly and may be in vivo, ex vivo or in vitro. Unless otherwise indicated each embodiment as described herein may be combined with another embodiment as described herein.

The sequence information as provided herein should not be so narrowly construed as to require inclusion of erroneously identified bases. The skilled person can identify such erroneously identified bases and knows how to correct for such errors. All patent and literature references cited in the present specification are hereby incorporated by reference in their entirety.

### EXAMPLES

### Example 1. Providing and Testing Antisense Oligonucleotides (AONs) for Efficient Skipping of Exon 62 in Human USH2A Pre-mRNA

The sequence of exon 62 of the human USH2A gene and its surrounding intron sequences were analyzed for the presence of exonic splice enhancer (ESE) motifs. Multiple sites were initially determined and forty-eight antisense oligonucleotides (AON Ex62.1 to AON Ex62.22; SEQ ID NO: 1 to 22, respectively; and AON Ex62.23 to AON Ex62.48; SEQ ID NO: 33 to 58, respectively) were manufactured in-house based on these ESE findings. Initially all AONs were modified with a 2′-O-methoxyethyl (2′-MOE) group at the sugar chain and all had a full phosphorothioated (PS) backbone. AONs were kept dissolved in PBS. The 3′ to 5′ sequences of all AONs are given in FIG. 1, under the target sequence of exon 62 of the human USH2A gene (this is given in FIG. 1 as DNA, but the skilled person is aware of the fact that the target is the corresponding pre-mRNA), and part of the upstream and downstream intron sequences. As becomes clear in FIG. 1, some AONs are partly complementary to an exon sequence at the 5′ end of exon 62, overlap the intron/exon boundary and are partly complementary to the upstream intron 61 (such as Ex62.17, Ex62.18 and Ex62.19), while other AONs are complementary to a sequence that is completely within exon 62, while yet other AONs are partly complementary to an exon sequence at the 3′ end of exon 62, overlap the exon/intron boundary and are partly complementary to a sequence of intron 62 (such as Ex62.20, Ex62.21 and Ex62.22).

To test the ability of the AONs to skip exon 62 from human USH2A pre-mRNA, the following procedures were performed.

### Cell Culture and Transfection

The WERI-Rb1 (ATCC® HTB-169™) retinoblastoma cell line was obtained from ATCC. Cells were cultured in RPMI 1640 medium (Gibco) supplemented with 10% FBS. WERI-Rb1 is a suspension cell line and was maintained by addition of fresh medium or replacement of medium every 3 to 4 days. When passaging the cells, the concentration of the cells was kept at 3×10⁵ cells per mL, at 37° C. and 5% CO₂.

For transfection, cells were seeded at a concentration of 4×10⁵ cells in 3,8 cm² wells in 0.9 mL RPMI 1640 supplemented with 10% FBS in a 12-well plate. The next day, cells were transfected with 50 nM of each oligonucleotide applied using Lipofectamine 2000 transfection reagent (Invitrogen). As a negative control, non-transfected (NT) and mock transfected cells were taken along. A ratio of 2:1 (volume/weight) between Lipofectamine 2000 and the AON was used. Both Lipofectamine 2000 and AON were prepared in Opti-MEM. Per condition, 50 μL of the Lipofectamine 2000 mixture was added to 50 μL AON mixture and incubated for 20 min at RT before adding the transfection complexes to the cells. Cells were incubated for 48 h at 37° C. Transfections were performed in triplicate.

### RNA Isolation and cDNA Synthesis

Total RNA was isolated from the cells using the RNeasy Plus Mini Kit (Qiagen) according to the manufacturer's protocol. RNA was eluted in 40 μL RNase free water and the concentrations were measured on the Nanodrop 2000. Samples were stored at −80° C. cDNA was synthesized using 300 ng total RNA. A 20 μL reaction contained 1 μL Verso Reverse Transcriptase enzyme, 4 μL 5×cDNA buffer, 2 μL dNTP mix [5 mM], 1 μL of the RT enhancer and 1 μL Random Hexamer Primers [400 ng/μL] (Thermo Scientific). The reaction was run in a thermocycler for 30 min at 42° C., 2 minutes at 95° C. and kept at 4-12° C. Samples were stored at −20° C.

### ddPCR Analysis

For the quantification of USH2A Δexon 62, ddPCR was performed with 60 ng WERI-Rb1 mRNA using ddPCR supermix for probes (no dUTP) (Bio-Rad) in a multiplex manner. The final 21 μL reaction mix contained 10.5 μL Supermix, 250 nM USH2A Δexon 62 forward and reverse primer, 90 nM USH2A Δexon 62 (FAM) probe and 600 nM of the USH2A Exon 50 reference assay. Primer and probe sequences are summarized below. The exon 62 forward primer is SEQ ID NO: 27. The exon 62 reverse primer is SEQ ID NO: 28. The exon 62 probe is SEQ ID NO: 29. The exon 50 forward primer is SEQ ID NO: 30. The exon 50 reverse primer is SEQ ID NO: 31. The exon 50 probe is SEQ ID NO: 32.

| USH2A ΔExon | |
|---|---|
| 62 assay | Sequence |
| Forward primer | 5′-GAC CCG ACG ATC CTA CAT-3′ |
| Reverse primer | 5′-GCG GAA GAG AAA CTG ACG-3′ |
| Probe sequence | 5′/56-FAM/CACAGTGAA/ZEN/GACAT |
| | ACAACATCTTCAGTGACGG/3IABkFQ/-3′ |
| USH2A Exon 50 | |
| reference assay | Sequence |
| Forward primer | 5′-CAG ATT TGC TGT GCT GGG AG-3′ |
| Reverse primer | 5′-TTC ACA TAA TCC TGC CCA CA-3′ |
| Probe sequence | 5′-/5HEX/CATACCTGGAAGGCGATTGTACA |
| | CCACTC/3IABkFQ/-3′ |

PCR reactions were dispersed into droplets using the QX200 droplet generator (Bio-Rad) according to the manufacturer's instructions and transferred to a 96-well PCR plate. End point PCR was performed in a T100 Thermocycler (Bio-Rad). The ddPCR protocol was as follows: denaturation at 94° C., annealing/extension at 61° C. in 40 cycles, enzyme deactivation at 98° C. and kept indefinitely at 4° C. till further analysis. The fluorescence of each droplet was quantified in the QX200 droplet reader (Bio-Rad). Each sample was analyzed in duplicate. Absolute quantification was performed in QuantaSoft software (Bio-Rad). Thresholds were manually set to distinguish between positive and negative droplets.

The primary analysis was performed using the QuantaSoft software. Only samples were included for further analysis when the total number of droplets was 10.000 per well. The negative control samples were checked for any application. The accepted samples were checked for both USH2A Exon 50 reference values represented by the green (HEX) color and for USH2A Exon 62 skipped values represented by the blue (FAM) droplets. Gating was performed manually separating the positive fluorescent cloud of droplets from the negative fluorescent droplets. After gating, the positive droplet counts in copies/20 μL for the two replicates was transported to an Excel file for secondary analysis. First the total copy numbers per sample for the two technical duplicates of each sample were averaged. Next the percentage skip was calculated by dividing the copies/20 μL found with the exon 62 skip assay by those detected with the exon 50 reference assay times 100. Finally, the percentage skip per AON was calculated by averaging the three separate performed replicates and the standard error of mean (SEM) was derived from these final values.

The final percentages of exon 62 skip from the human wild type USH2A pre-mRNA and the SEM, using the first 22 AONs as depicted on the x-axis (distributed according to their target area in exon 62 and its surrounding intron sequences, are depicted in FIG. 2. This shows that the background skip in the untreated sample was low (2-3% background) and that all tested AONs gave a higher skip percentage then the untreated controls. Three areas that were initially tested here showed higher skip percentages, with two AONs at the 5′ end of exon 62 outperforming other AONs (AON Ex62.18 with 18% and AON Ex62.13 with 13% of exon 62 skip), and at the 3′ end of exon 62 (AON Ex62.22 with 8%).

### Example 2. Testing AONs for Improved Exon 62 Skipping in Human USH2A Pre-mRNA

A second generation of oligonucleotides was designed around the three areas with the highest skip percentages (represented by AONs Ex62.18, Ex62.13 and Ex62.22, see above) for a subsequent test and ddPCR analysis that were performed according to the procedures outlined in example 1. New AONs Ex62.23, -24, 25, and -26 were in the area of AON Ex62.18. New AONs Ex62.27, -28, and -29 were in the area of AON Ex62.13. New AONs Ex62.30 and -31 were in the area of AON Ex62.22.

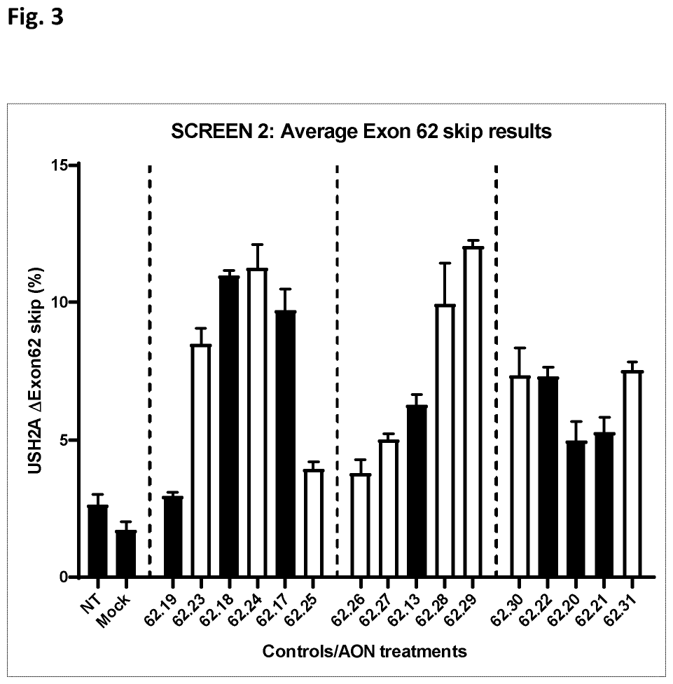

The results with these and earlier manufactured AONs are given in FIG. 3, which indicates that a few new AONs outperformed the earlier developed AONs, such as AON Ex62.24 that performed better than AON Ex62.18, and AON Ex62.28 and -29 that outperformed AON Ex62.13 using transfection.

### Example 3. Testing AONs for Improved Exon 62 Skipping in Human USH2A Pre-mRNA

Then, a third set of AONs was generated in the areas surrounding AON Ex62.24 and AON Ex62.29 that performed best in the second screen (example 2, FIG. 2). The inventors asked themselves whether shorter AONs would perform better than the longer ones. AON Ex62.24 is a 23-mer, while AON Ex62.28 and -29 are both 24-mers. The transfection procedures in WERI-Rb1 cells with AON Ex62.32 (19-mer), -33 (18-mer), -34 (17-mer), -35 (16-mer), -36 (17-mer), -37 (18-mer), and -38 (19-mer) in the area of AON Ex62.24, and with AON Ex62.39 (22-mer), -40 (21-mer), -41 (19-mer), -42 (18-mer), and -43 (17-mer) in the area of AON Ex62.28 and -29, were as described above.

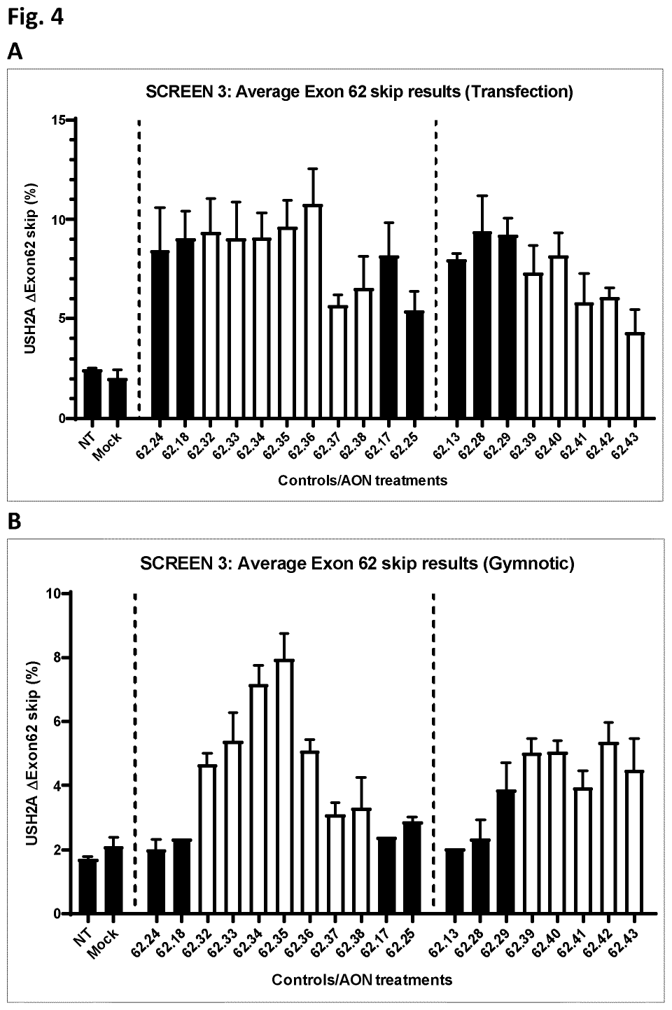

The results with these and earlier manufactured AONs from this transfection assay are given in FIG. 4A, which indicates that a few new AONs outperformed the earlier developed AONs. AON Ex62.36 (a 17-nt containing oligonucleotide) performed best in this experiment.

The nineteen AONs that were tested in their ability to generate exon 62 skip from human USH2A pre-mRNA after transfection were then tested in an experimental setup that was considered to represent a clinical setting better, namely without using transfection reagents. It was envisioned that after naked delivery of the oligonucleotides by direct intravitreal injection in the eye, the AONs must reach the retinal cells and enter those without additives such as transfection reagents, nanoparticles or (viral) vectors. Hence, it was realized that an AON that would provide the best skip in an in vitro transfection assay could potentially be outperformed by an AON that would be delivered without transfection reagents and be better suited to be tested in a clinical setting in vivo. Such direct delivery method is generally referred to as ‘gymnotic uptake’. The gymnotic uptake experiment with 10 μM of each AON on WERI-Rb1 cells was performed generally as follows: Cells were seeded at a concentration of 5×10⁵ cell s per well in 3,8 cm² wells in 0.9 mL RPMI 1640 supplemented with 10% FBS in a 12-well plate. The next day cells were treated with 10 μM of each oligonucleotide by adding the “naked” AONs to the medium. As a negative control, non-treated (NT) cells were taken along. Cells were incubated for 65 h at 37° C. This gymnotic uptake experiment of each AON was performed in triplicate.

The results of these experiments are shown in FIG. 4B. Strikingly, the larger AONs that performed quite well in the transfection assay (such as AON EX62.18 and Ex62.24) did not give exon 62 skipping above background levels when tested under gymnotic uptake conditions. However, the shortest AON in the set covering the same exon 62 area, namely AON Ex62.35, which is a 16-mer, gave the highest skipping percentage in the one region, and AON Ex62.42 (an 18-mer) in the other. It does not seem illogical that a short oligonucleotide can enter cells or traffic through cells towards and enter the nucleus (on its own) better than a long oligonucleotide, although it remains to be determined how stable these AONs of different length are in in vivo situations. Importantly, the skilled person understands that there is a lower limit to an oligonucleotide as far as specificity goes. This needs to be assessed per target, per sequence and per genome, because for each oligonucleotide sequence a potential off-target complementarity sequence may or may not exist in the (human) genome. But even though a complementary sequence may exist somewhere else in a genome, such may not hamper the development of a therapeutic, depending where such ‘other’ complementary sequence is located. Of course, it will be appreciated by the skilled person that gymnotic uptake is not the only measure for determining whether an AON is suited for its purpose or not. It may be that there are immunological issues, Tm specifics, and half-life differences. It may also be that an AON that does not enter the cell and/or nucleus after gymnotic uptake assessment is very suited for exon 62 skipping for instance when delivered in another way, such as through (viral) vectors or nanoparticles, or when it is chemically modified or introduced in target cells in another way.

### Example 4. Testing Short AONs for Improved Exon 62 Skipping in Human USH2A Pre-mRNA after Gymnotic Uptake

The finding that short AONs could outperform long AONs after gymnotic uptake as described in example 3 was further assessed by manufacturing a number of additional oligonucleotides: AON Ex62.44 (22-mer), -45 (20-mer), -46 (18-mer), -47 (16-mer), and -48 (14-mer). See FIG. 1 for their positions in relation to their target sequence. These and earlier used AONs were used in a gymnotic uptake assay using WERE-Rb1 cells and ddPCR on the resulting RNA, as outlined above.

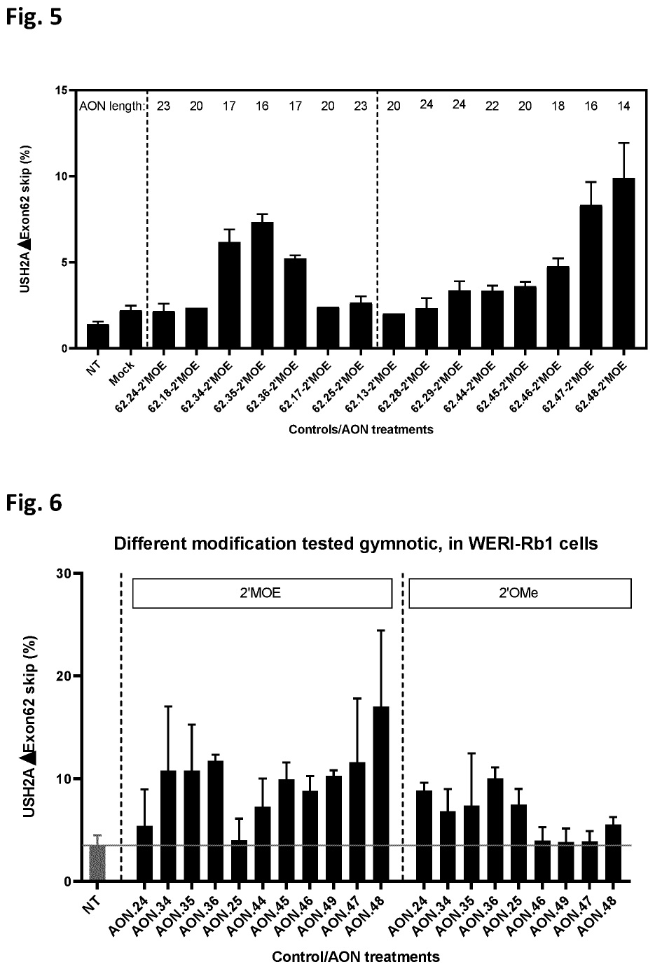

The results are depicted in FIG. 5, and show that the shortest oligonucleotide, AON Ex62.48 which consists of only 14 nucleotides outperformed all other tested AONs, with the 16-mer AON Ex62.47 as the best runner-up. Also, AON Ex62.34 (a 17-mer) and AON Ex62.35 (a 16-mer) gave significant exon 62 skipping.

### Example 5. Testing Different 2′ Modifications

Further to the experiments outlined above, several good performing AONs and control AONs were manufactured in a 2′-O-Methyl (2′OMe) modified form to test in gymnotic uptake in WERI-Rb1 cells, generally using the methods described above. Results are shown in FIG. 6, which shows on the left the results with the 2′MOE AONs and on the right side the results with a number of corresponding AONs that were modified with 2′-OMe. AON Ex62.49 (SEQ ID NO: 60) was a newly tested AON. FIG. 1 shows additional AONs in this particular region: AON Ex62.50 (SEQ ID NO:61), AON Ex62.51 (SEQ ID NO: 62) and AON Ex62.52 (SEQ ID NO: 63), that were not tested here, but that are likely to perform in a similar good fashion. Only AON Ex62.25 performed better with 2′-OMe than with 2′-MOE. This shows that—in general—a 2′-MOE modified AON is preferred at least when applying these gymnotic uptake experiments to skip exon 62.

### Example 6. Dose-Response Testing of Best Performing AONs

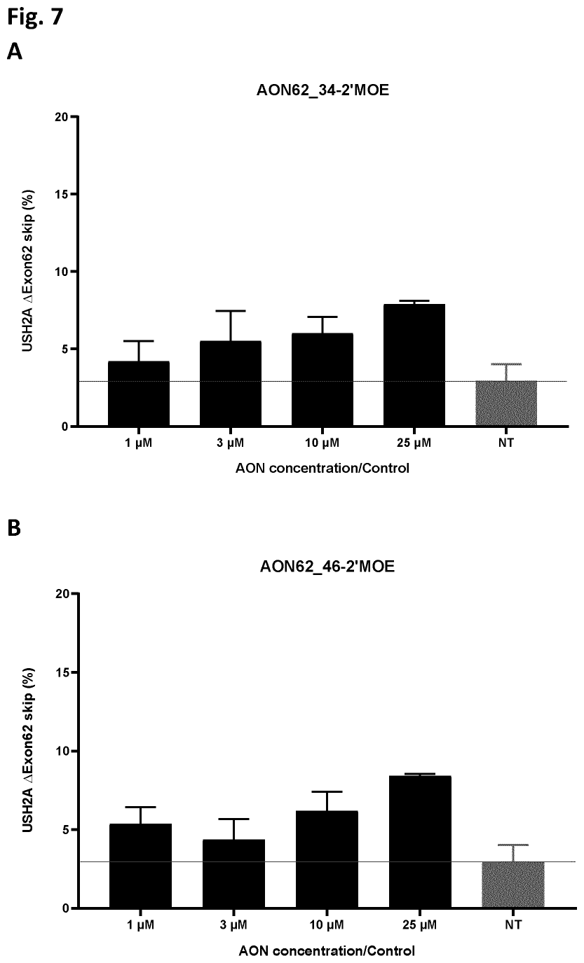

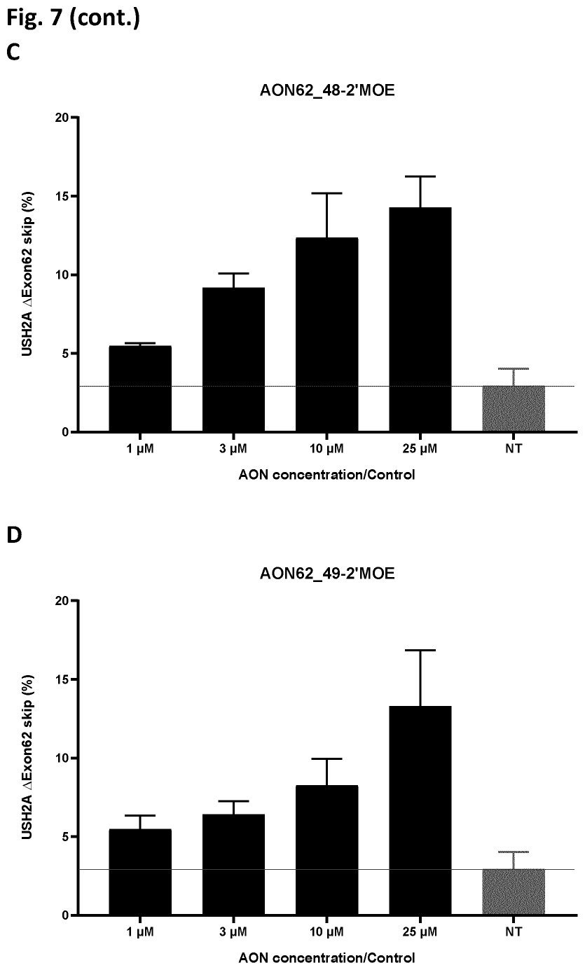

AONs Ex62.34, -46, -48 and -49 (all 2′-MOE modified) were tested in a dose response gymnotic uptake experiment in triplicate in WERI-Rb1 cells. Methods were generally as described above. Screening was performed with 1, 3, 10 and 25 μM AON. Average results are plotted in FIG. 7, which shows that especially AONs Ex62.48 and -49 exhibited a clear dose-response, with high percentages of exon 62 skip from the human USH2A pre-mRNA.

### Example 7. Testing AONs for Exon 62 Skipping in Human Organoids

Wild-type induced pluripotent stem cells (iPSC) were differentiated into retinal organoids and cultured for approximately 180 days using a differentiation protocol based on the methods as described by Hallam et al. (2018. Stem Cells 36(10):1531-1551) and Kuwahara et al. (2015. Nat Commun 6:6286). After differentiation, organoids were separately treated with AONs Ex62.34, -46, -48 and -49 (0.3 or 7.5 μM; all 2′-MOE modified; for 14 days). As a control, separate organoids were treated with 7.5 μM unrelated control AON for 14 days. Every other day, half of the culture medium was refreshed with fresh culture medium containing AONs. After 14 days, organoids were collected, and RNA was extracted using Direct-zol RNA Microprep kit (Zymo Research) using the recommendations of the manufacturer. cDNA was synthesized with 150 ng RNA using the Verso cDNA synthesis kit (Thermo Fisher Scientific) using the manufacturer's protocol. A master mix was prepared containing 6 μL 5×cDNA synthesis buffer, 3 μL dNTP mix, 1.5 μL RT enhancer, 1.5 μL random hexamer primers and 1.5 μL Verso enzyme mix per sample (30 μL in total). Reactions were incubated at 42° C. for 30 min and heat-inactivated at 95° C. for 2 min. For mRNA quantification of exon 62 skip, ddPCR was performed using 20 ng cDNA to analyze USH2A exon 62 wild-type, exon 62 skip and exon 50 reference (not skipped). In addition, levels of the retinal marker CRX (Hs00230899_m1, Thermo Fisher Scientific) was measured in the organoids to show that they were well-differentiated (data not shown). USH2A exon 62 skip percentage was calculated by the following formula:

$$Skip percentage = \frac{( Δ exon 62 ) sample}{( Exon 50 ) sample} \times \frac{( Exon 50 ) control}{( Exon 62 + Δ exon 62 ) control} \times 100$$

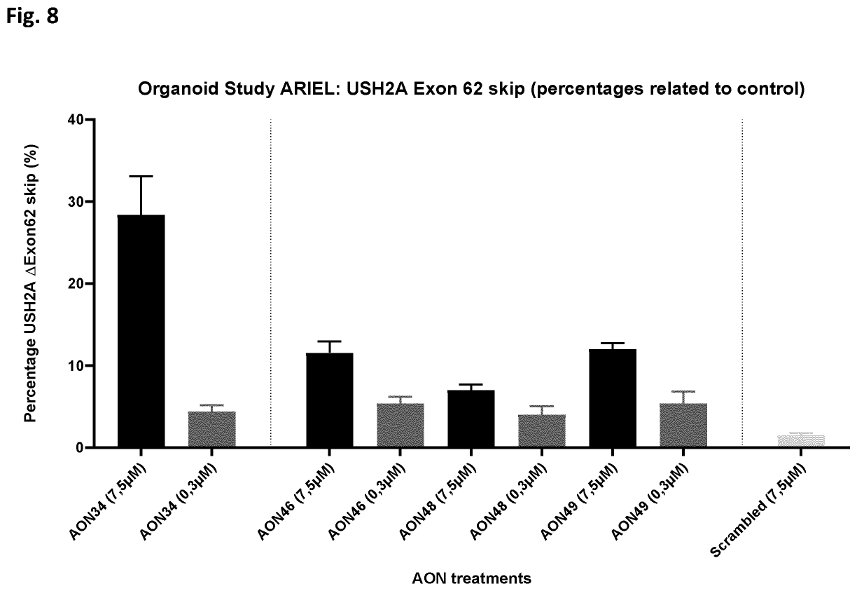

The final average percentages of exon 62 skip from the human wild type USH2A pre-mRNA and the SEM are shown in FIG. 8 and clearly indicate that administration of the best performing oligonucleotide, AON Ex62.34, gives a skip percentage of 28% in human organoids using a concentration of 7.5 μM. This clearly shows that the inventors of the present invention were capable of achieving a significant skip effect in human material that represents a retina, indicating that using an AON for skipping exon 62 from human USH2A pre-mRNA is a feasible concept providing means to treat Usher syndrome in human subjects, where the syndrome is caused by mutations present in exon 62 of the subject's USH2A gene.

1. An antisense oligonucleotide (AON) capable of skipping exon 62 from human USH2A pre-mRNA, wherein the AON under physiological conditions binds to and/or is complementary to a sequence of SEQ ID NO: 25 or 26, or a part thereof.

2. The AON according to claim 1, wherein the AON under physiological conditions binds to and/or is complementary to a sequence of SEQ ID NO: 25 that includes the 5′ intron/exon boundary of exon 62, wherein the AON under physiological conditions binds to and/or is complementary to a sequence of SEQ ID NO: 25 that is completely within exon 62, or wherein the AON under physiological conditions binds to and/or is complementary to a sequence of SEQ ID NO: 25 that includes the 3′ exon/intron boundary of exon 62.

3. The AON according to claim 1, wherein the AON comprises or consists of, the sequence of SEQ ID NO: 1, 2, 3, 4, 5, 6, 7, 8, 9, 10, 11, 12, 13, 14, 15, 16, 17, 18, 19, 20, 21, 22, 33, 34, 35, 36, 37, 38, 39, 40, 41, 42, 43, 44, 45, 46, 47, 48, 49, 50, 51, 52, 53, 54, 55, 56, 57, 58, 60, 61, 62, or 63.

4. The AON according to claim 1, wherein the AON consists of from 8 to 143 nucleotides, and preferably consists of 14, 15, 16, 17, 18, 19, 20, 21, 22, 23, or 24 nucleotides.

5. The AON according to claim 1, wherein the AON is an oligoribonucleotide.

6. The AON according to claim 1, wherein the AON comprises at least one 2′-O-methoxyethyl (2′-MOE) modification, preferably wherein all nucleotides of the AON are 2′-MOE modified.

7. The AON according to claim 1, wherein the AON comprises at least one non-naturally occurring internucleosidic linkage, such as a phosphorothioate (PS) linkage, preferably wherein all sequential nucleosides are interconnected by PS linkages.

8. A viral vector expressing an AON according to claim 1.

9. A pharmaceutical composition comprising an AON according to claim 1 and a pharmaceutically acceptable carrier.

10. The AON according to claim 1 use in the treatment, prevention or delay of an USH2A-related disease or a condition requiring modulating splicing of USH2A pre-mRNA, such as Usher syndrome type II caused by a mutation in exon 62.

11. The AON for use according to claim 10, wherein the AON is for intravitreal administration and is dosed in an amount ranging from 5 μg to 500 μg of total AON per eye, such as about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, or 320 μg total AON per eye.

12. Use of an AON according to claim 1 for the preparation of a medicament for the treatment, prevention or delay of an USH2A-related disease or a condition requiring modulating splicing of USH2A pre-mRNA, such as Usher syndrome type II caused by a mutation in exon 62.

13. An in vitro, ex vivo or in vivo method for modulating splicing of USH2A pre-mRNA in a cell, comprising the steps of: administering to the cell an AON according claim 1; allowing the hybridization of the AON to its complementary sequence in USH2A target RNA molecule in the cell; and allowing the skip of exon 62 from the target RNA molecule.

14. A method for the treatment of a USH2A-related disease or condition requiring modulating splicing of USH2A pre-mRNA of an individual in need thereof, said method comprising contacting a cell of said individual with an AON according to claim 1.

15. A pharmaceutical composition comprising a viral vector according to claim 8 and a pharmaceutically acceptable carrier.

16. The viral vector according to claim 8 for use in the treatment, prevention or delay of an USH2A-related disease or a condition requiring modulating splicing of USH2A pre-mRNA, such as Usher syndrome type II caused by a mutation in exon 62.

17. The pharmaceutical composition according to claim 9 for use in the treatment, prevention or delay of an USH2A-related disease or a condition requiring modulating splicing of USH2A pre-mRNA, such as Usher syndrome type II caused by a mutation in exon 62.

18. Use of a viral vector according to claim 8 for the preparation of a medicament for the treatment, prevention or delay of an USH2A-related disease or a condition requiring modulating splicing of USH2A pre-mRNA, such as Usher syndrome type II caused by a mutation in exon 62.

19. Use of a pharmaceutical composition according to claim 9 for the preparation of a medicament for the treatment, prevention or delay of an USH2A-related disease or a condition requiring modulating splicing of USH2A pre-mRNA, such as Usher syndrome type II caused by a mutation in exon 62.

20. An in vitro, ex vivo or in vivo method for modulating splicing of USH2A pre-mRNA in a cell, comprising the steps of: administering to the cell a viral vector according to claim 8; allowing the hybridization of the AON to its complementary sequence in USH2A target RNA molecule in the cell; and allowing the skip of exon 62 from the target RNA molecule.

21. An in vitro, ex vivo or in vivo method for modulating splicing of USH2A pre-mRNA in a cell, comprising the steps of: administering to the cell a pharmaceutical composition according to claim 9; allowing the hybridization of the AON to its complementary sequence in USH2A target RNA molecule in the cell; and allowing the skip of exon 62 from the target RNA molecule.

22. A method for the treatment of a USH2A-related disease or condition requiring modulating splicing of USH2A pre-mRNA of an individual in need thereof, said method comprising contacting a cell of said individual with a viral vector according to claim 8.

23. A method for the treatment of a USH2A-related disease or condition requiring modulating splicing of USH2A pre-mRNA of an individual in need thereof, said method comprising contacting a cell of said individual with a pharmaceutical composition according to claim 9.


SEQUENCE LISTING (63 sequences)

SEQ ID NO: 1 - RNA, 21 nt, Artificial sequence
  AON Ex62.1
agggcuuaaaauuucuccugc

SEQ ID NO: 2 - RNA, 21 nt, Artificial sequence
  AON Ex62.2
ccaagggcuuaaaauuucucc

SEQ ID NO: 3 - RNA, 21 nt, Artificial sequence
  AON Ex62.3
uuugaaucagaguccaagggc

SEQ ID NO: 4 - RNA, 21 nt, Artificial sequence
  AON Ex62.4
aaguuucucaguccacuuggg

SEQ ID NO: 5 - RNA, 21 nt, Artificial sequence
  AON Ex62.5
ccggccauucucuuucuguuc

SEQ ID NO: 6 - RNA, 20 nt, Artificial sequence
  AON Ex62.6
augcccggccauucucuuuc

SEQ ID NO: 7 - RNA, 20 nt, Artificial sequence
  AON Ex62.7
ugaccacuguaguagcaaug

SEQ ID NO: 8 - RNA, 20 nt, Artificial sequence
  AON Ex62.8
acaccauugguucucauagg

SEQ ID NO: 9 - RNA, 20 nt, Artificial sequence
  AON Ex62.9
augguuugcagccacaacac

SEQ ID NO: 10 - RNA, 20 nt, Artificial sequence
  AON Ex62.10
ccacaacaccaaugcgauau

SEQ ID NO: 11 - RNA, 20 nt, Artificial sequence
  AON Ex62.11
gcaugguuugcagccacaac

SEQ ID NO: 12 - RNA, 20 nt, Artificial sequence
  AON Ex62.12
cuuggggaagauucuaaggu

SEQ ID NO: 13 - RNA, 20 nt, Artificial sequence
  AON Ex62.13
augguucuaacccguacagg

SEQ ID NO: 14 - RNA, 20 nt, Artificial sequence
  AON Ex62.14
ugggcuugauggcuuguucc

SEQ ID NO: 15 - RNA, 24 nt, Artificial sequence
  AON Ex62.15
cggccauucucuuucuguucuacu

SEQ ID NO: 16 - RNA, 24 nt, Artificial sequence
  AON Ex62.16
auaaaguuucucaguccacuuggg

SEQ ID NO: 17 - RNA, 20 nt, Artificial sequence
  AON Ex62.17
gauggcuuguucccuguaag

SEQ ID NO: 18 - RNA, 20 nt, Artificial sequence
  AON Ex62.18
ggcuuguucccuguaagaaa

SEQ ID NO: 19 - RNA, 24 nt, Artificial sequence
  AON Ex62.19
guucccuguaagaaaauuaacagg

SEQ ID NO: 20 - RNA, 24 nt, Artificial sequence
  AON Ex62.20
gguagacauuaccuuaaucacacc

SEQ ID NO: 21 - RNA, 23 nt, Artificial sequence
  AON Ex62.21
gcaggguagacauuaccuuaauc

SEQ ID NO: 22 - RNA, 24 nt, Artificial sequence
  AON Ex62.22
gacauuaccuuaaucacaccauug

SEQ ID NO: 23 - DNA, 294 bp, Homo sapiens
aaaaaaaaaaaacaacaacttaacctgttaattttcttacagggaacaagccatcaagcccacctgtacgggttagaaccattcacaacatatcgcattggtgttgtggctgcaaaccatgcaggagaaattttaagcccttggactctgattcaaaccttagaatcttccccaagtggactgagaaactttatagtagaacagaaagagaatggccgggcattgctactacagtggtcagaacctatgagaaccaatggtgtgattaaggtaatgtctaccctgcataagaaa

SEQ ID NO: 24 - DNA, 228 bp, Homo sapiens
ggaacaagccatcaagcccacctgtacgggttagaaccattcacaacatatcgcattggtgttgtggctgcaaaccatgcaggagaaattttaagcccttggactctgattcaaaccttagaatcttccccaagtggactgagaaactttatagtagaacagaaagagaatggccgggcattgctactacagtggtcagaacctatgagaaccaatggtgtgattaag

SEQ ID NO: 25 - RNA, 294 nt, Homo sapiens
aaaaaaaaaaaacaacaacuuaaccuguuaauuuucuuacagggaacaagccaucaagcccaccuguacggguuagaaccauucacaacauaucgcauugguguuguggcugcaaaccaugcaggagaaauuuuaagcccuuggacucugauucaaaccuuagaaucuuccccaaguggacugagaaacuuuauaguagaacagaaagagaauggccgggcauugcuacuacaguggucagaaccuaugagaaccaauggugugauuaagguaaugucuacccugcauaagaaa

SEQ ID NO: 26 - RNA, 228 nt, Homo sapiens
ggaacaagccaucaagcccaccuguacggguuagaaccauucacaacauaucgcauugguguuguggcugcaaaccaugcaggagaaauuuuaagcccuuggacucugauucaaaccuuagaaucuuccccaaguggacugagaaacuuuauaguagaacagaaagagaauggccgggcauugcuacuacaguggucagaaccuaugagaaccaauggugugauuaag

SEQ ID NO: 27 - DNA, 18 bp, Artificial sequence
  ddPCR forward primer
gacccgacgatcctacat

SEQ ID NO: 28 - DNA, 18 bp, Artificial sequence
  ddPCR reverse primer
gcggaagagaaactgacg

SEQ ID NO: 29 - DNA, 33 bp, Artificial sequence
  exon 62 FAM probe
cacagtgaagacatacaacatcttcagtgacgg

SEQ ID NO: 30 - DNA, 20 bp, Artificial sequence
  ddPCR forward primer
cagatttgctgtgctgggag

SEQ ID NO: 31 - DNA, 20 bp, Artificial sequence
  ddPCR reverse primer
ttcacataatcctgcccaca

SEQ ID NO: 32 - DNA, 29 bp, Artificial sequence
  exon 50 HEX probe
catacctggaaggcgattgtacaccactc

SEQ ID NO: 33 - RNA, 23 nt, Artificial sequence
  AON Ex62.23
ggcuuguucccuguaagaaaauu

SEQ ID NO: 34 - RNA, 23 nt, Artificial sequence
  AON Ex62.24
gauggcuuguucccuguaagaaa

SEQ ID NO: 35 - RNA, 23 nt, Artificial sequence
  AON Ex62.25
cuugauggcuuguucccuguaag

SEQ ID NO: 36 - RNA, 24 nt, Artificial sequence
  AON Ex62.26
guucuaacccguacaggugggcuu

SEQ ID NO: 37 - RNA, 24 nt, Artificial sequence
  AON Ex62.27
augguucuaacccguacagguggg

SEQ ID NO: 38 - RNA, 24 nt, Artificial sequence
  AON Ex62.28
gugaaugguucuaacccguacagg

SEQ ID NO: 39 - RNA, 24 nt, Artificial sequence
  AON Ex62.29
guugugaaugguucuaacccguac

SEQ ID NO: 40 - RNA, 24 nt, Artificial sequence
  AON Ex62.30
accuuaaucacaccauugguucuc

SEQ ID NO: 41 - RNA, 23 nt, Artificial sequence
  AON Ex62.31
uuaugcaggguagacauuaccuu

SEQ ID NO: 42 - RNA, 19 nt, Artificial sequence
  AON Ex62.32
ggcuuguucccuguaagaa

SEQ ID NO: 43 - RNA, 18 nt, Artificial sequence
  AON Ex62.33
ggcuuguucccuguaaga

SEQ ID NO: 44 - RNA, 17 nt, Artificial sequence
  AON Ex62.34
gcuuguucccuguaaga

SEQ ID NO: 45 - RNA, 16 nt, Artificial sequence
  AON Ex62.35
gcuuguucccuguaag

SEQ ID NO: 46 - RNA, 17 nt, Artificial sequence
  AON Ex62.36
ggcuuguucccuguaag

SEQ ID NO: 47 - RNA, 18 nt, Artificial sequence
  AON Ex62.37
uggcuuguucccuguaag

SEQ ID NO: 48 - RNA, 19 nt, Artificial sequence
  AON Ex62.38
auggcuuguucccuguaag

SEQ ID NO: 49 - RNA, 22 nt, Artificial sequence
  AON Ex62.39
ugugaaugguucuaacccguac

SEQ ID NO: 50 - RNA, 21 nt, Artificial sequence
  AON Ex62.40
gugaaugguucuaacccguac

SEQ ID NO: 51 - RNA, 19 nt, Artificial sequence
  AON Ex62.41
gaaugguucuaacccguac

SEQ ID NO: 52 - RNA, 18 nt, Artificial sequence
  AON Ex62.42
aaugguucuaacccguac

SEQ ID NO: 53 - RNA, 17 nt, Artificial sequence
  AON Ex62.43
augguucuaacccguac

SEQ ID NO: 54 - RNA, 22 nt, Artificial sequence
  AON Ex62.44
guugugaaugguucuaacccgu

SEQ ID NO: 55 - RNA, 20 nt, Artificial sequence
  AON Ex62.45
guugugaaugguucuaaccc

SEQ ID NO: 56 - RNA, 18 nt, Artificial sequence
  AON Ex62.46
guugugaaugguucuaac

SEQ ID NO: 57 - RNA, 16 nt, Artificial sequence
  AON Ex62.47
guugugaaugguucua

SEQ ID NO: 58 - RNA, 14 nt, Artificial sequence
  AON Ex62.48
guugugaaugguuc

SEQ ID NO: 59 - DNA, 274 bp, Homo sapiens
taacctgttaattttcttacagggaacaagccatcaagcccacctgtacgggttagaaccattcacaacatatcgcattggtgttgtggctgcaaaccatgcaggagaaattttaagcccttggactctgattcaaaccttagaatcttccccaagtggactgagaaactttatagtagaacagaaagagaatggccgggcattgctactacagtggtcagaacctatgagaaccaatggtgtgattaaggtaatgtctaccctgcataagaaa

SEQ ID NO: 60 - RNA, 17 nt, Artificial sequence
  AON Ex62.49
guugugaaugguucuaa

SEQ ID NO: 61 - RNA, 21 nt, Artificial sequence
  AON Ex62.50
guugugaaugguucuaacccg

SEQ ID NO: 62 - RNA, 19 nt, Artificial sequence
  AON Ex62.51
guugugaaugguucuaacc

SEQ ID NO: 63 - RNA, 15 nt, Artificial sequence
  AON Ex62.52
guugugaaugguucu